(19) United States
(12) Patent Application Publication
(10) Pub. No.: US 2013/0131769 A1
(43) Pub. Date: May 23, 2013

(54) PRINTED CIRCUIT BOARD CONNECTION TO FEEDTHROUGH

(71) Applicants: Alexander K. Smith, Chesterland, OH (US); Daniel N. Kelsch, Fairview Park, OH (US)

(72) Inventors: Alexander K. Smith, Chesterland, OH (US); Daniel N. Kelsch, Fairview Park, OH (US)

(21) Appl. No.: 13/303,219

(22) Filed: Nov. 23, 2011

(51) Int. Cl. A61N 1/05; H05K 3/00

(52) U.S. Cl. 607/117; 29/829

(57) ABSTRACT

An implantable electronic device includes a housing wall defining an interior surface and an exterior surface. A feedthrough assembly includes a body coupled to the housing and defining an aperture, and a pin at least partially disposed within the aperture and passing through the housing wall from the interior surface to the exterior surface such that the pin has an interior portion and an exterior portion. A printed circuit board (PCB) has a substantially rigid portion defining a plane and a substantially flexible portion. The flexible portion has a distal end and a proximal end. The proximal end is coupled to the substantially rigid portion. The flexible portion is coupled to the pin interior portion adjacent the distal end. The flexible portion defines a bend between the proximal end and the distal end. At least one line tangent to the flexible portion is substantially perpendicular to the plane.

### BACKGROUND

The invention relates to an implantable pulse generator (IPG) of a stimulation system, such as a spinal cord stimulation (SCS) system.

A spinal cord stimulator is a pain-managing device used to provide electrical stimulation to the spinal cord or spinal nerve neurons. The stimulator includes an implantable pulse generator receiving an implanted medical electrical lead having one or more electrodes at a distal location thereof. Implantable pulse generators include electronic components coupled to a printed circuit board (PCB). The PCB is located within a hermetically sealed housing, or “can” of the IPG. Hermetically sealed feedthroughs connect the PCB to the electrodes. The invention relates to the connection between the PCB and the feedthroughs.

### SUMMARY

In one embodiment, the invention provides an implantable electronic device. A housing having a wall includes an interior surface and an exterior surface. A feedthrough assembly includes a body coupled to the housing and defining an aperture, and a pin at least partially disposed within the aperture and passing through the housing wall from the interior surface to the exterior surface such that the pin has an interior portion and an exterior portion. A printed circuit board (PCB) has a substantially rigid portion defining a plane and a substantially flexible portion. The flexible portion has a distal end and a proximal end. The proximal end is coupled to the substantially rigid portion. The flexible portion is coupled to the pin interior portion adjacent the distal end. The flexible portion defines a bend between the proximal end and the distal end, with at least one line tangent to the flexible portion being substantially perpendicular to the plane.

In another embodiment, the invention provides a method of assembling an implantable electronic device. A housing having an interior surface and an exterior surface is provided. A feedthrough assembly, including a body defining an aperture, and a pin at least partially disposed within the aperture and passing through body, is provided. The feedthrough assembly is coupled to the housing such that the pin extends through the housing to define an interior portion and an exterior portion. A printed circuit board (PCB) having a substantially rigid portion defining a plane and a substantially flexible portion is provided. A proximal end of the flexible portion is coupled to the rigid portion. A distal end of the flexible portion is coupled to the interior portion of the pin. The rigid portion is rotated about an axis substantially perpendicular to the pin. A bend is formed between the proximal end and the distal end, with at least one line tangent to the flexible portion being substantially perpendicular to the plane.

Other aspects of the invention will become apparent by consideration of the detailed description and accompanying drawings.

### BRIEF DESCRIPTION OF THE DRAWINGS

FIG. 1 is a partial perspective view of a patient using a spinal cord stimulation system.

FIG. 2 is a perspective view of an implantable pulse generator capable of being used in the system of FIG. 1.

FIG. 3 is a perspective view of a portion of the implantable pulse generator of FIG. 2, in a position of assembly.

FIG. 4 is a cross sectional view of a portion of the implantable pulse generator of FIG. 2.

FIG. 5 is a perspective view of a portion of the implantable pulse generator of FIG. 2.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways.

The invention herein relates to an electrical stimulation system for providing stimulation to target tissue of a patient. The system described in detail below relates to a spinal cord stimulation (SCS) system for providing electrical pulses to the neurons of the spinal cord of a patient. However, many aspects of the invention are not limited to spinal cord stimulation systems or components thereof. For example, the components, assemblies, and methods described herein may also used with deep brain stimulation systems, peripheral nerve stimulation systems, cochlear implants, retinal implant systems, artificial hearts, and prosthetic devices.

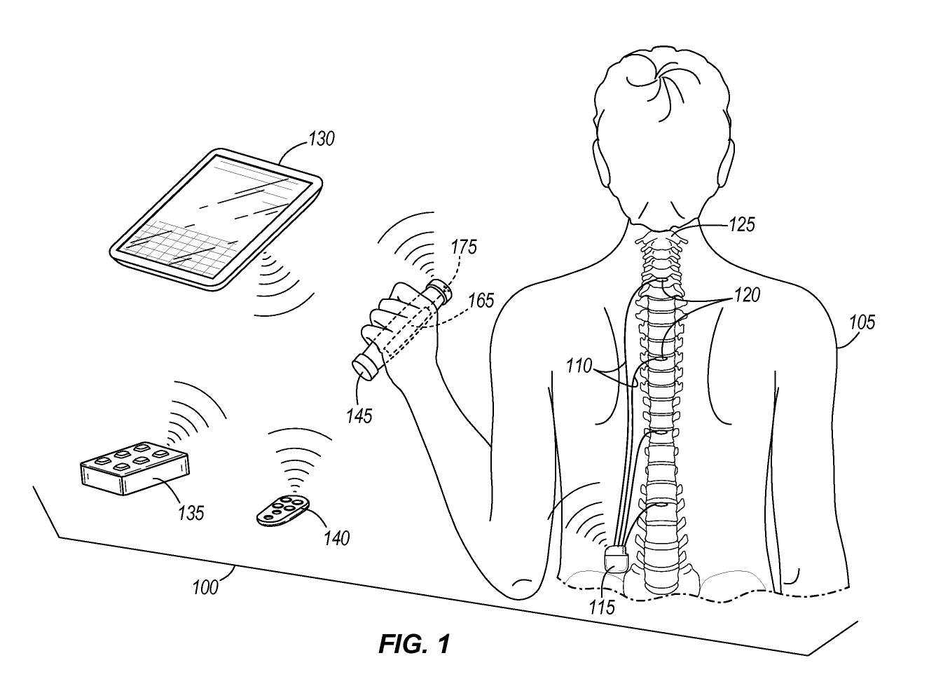

FIG. 1 shows a spinal cord stimulation system 100 in use with a patient 105. The system includes one or more implanted medical electrical leads 110 connected to an implantable pulse generator (IPG) 115. The leads 110 include an electrode array 120 at a distal end of the base lead cable. The electrode array 120 includes one or more electrical stimulation electrodes (may also be referred as electrode contacts or simply electrodes) and is placed adjacent to the dura of the spine 125 using an anchor. The spinal column includes the C1-C7 (cervical), T1-T12 (thoracic), L1-L5 (lumbar) and S1-S6 (sacral) vertebrae and the electrode array(s) 120 may be positioned anywhere along the spine 125 to deliver the intended therapeutic effects of spinal cord electrical stimulation in a desired region of the spine. The electrodes of the electrode arrays 120 promote electrical stimulation to the neurons of the spine based on electrical signals generated by the IPG 115. In one construction, the electrical signals are regulated current pulses that are rectangular in shape. However, the electrical signals can be other types of signals, including other types of pulses (e.g., regulated voltage pulses), and other shapes of pulses (e.g., trapezoidal, sinusoidal). The stimulation is provided from the IPG 115 to the electrodes via the base lead, which is connected to the IPG 115 with the proximal end of the base lead. The body of the lead can traverse through the body of the patient via the spinal column and from the spinal column through the body of the patient to the implant site of the IPG 115.

The IPG 115 generates the electrical signals through a multiplicity of electrodes (e.g., twenty seven electrodes). The IPG 115 can control six aspects of electrical stimulation based on a program (may also be referred to as a protocol): on/off, amplitude (e.g., current or voltage), frequency, pulse width, pulse shape, and polarity (anodic or cathodic stimulation. Typically, the IPG 115 is implanted in a surgically made pocket (e.g., in the abdomen) of the patient.

The IPG 115 communicates with any one of a clinician programmer (CP) 130, a patient programmer and charger (PPC) 135, and a pocket (or fob) programmer (PP) 140. A user provides feedback to the CP 130 with a PFD 145 while the CP 130 develops the protocol for the IPG 115.

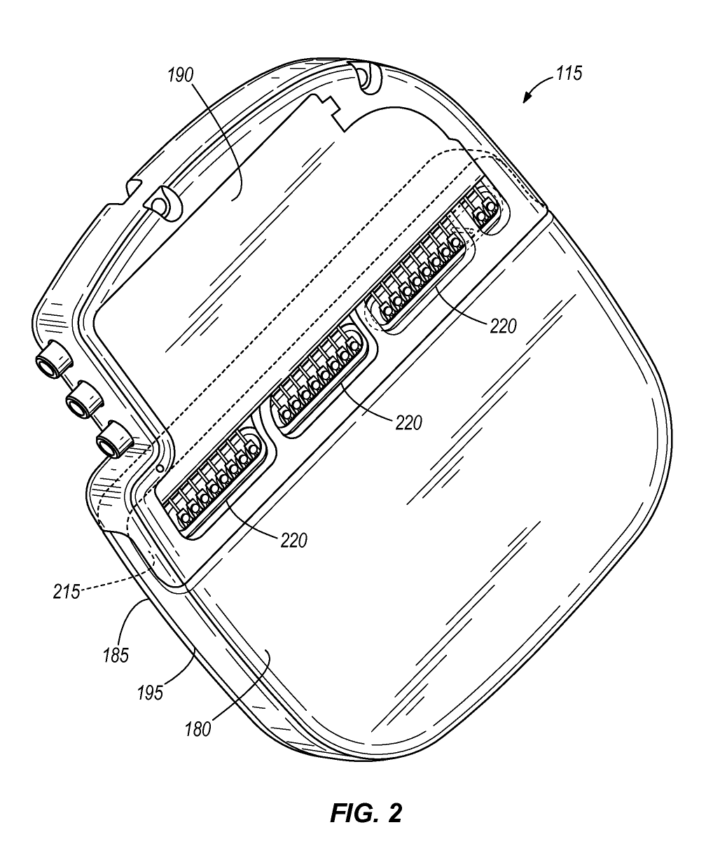

FIG. 2 illustrates a configuration of the IPG 115. The IPG 115 includes a first housing portion 180, a second housing portion 185, and a wire guide assembly 190. The first housing portion 180 and the second housing portion 185 are coupled along a hermetically sealed seam 195. Each of the first housing portion 180 and second housing portion 185 may be formed, for example, of titanium.

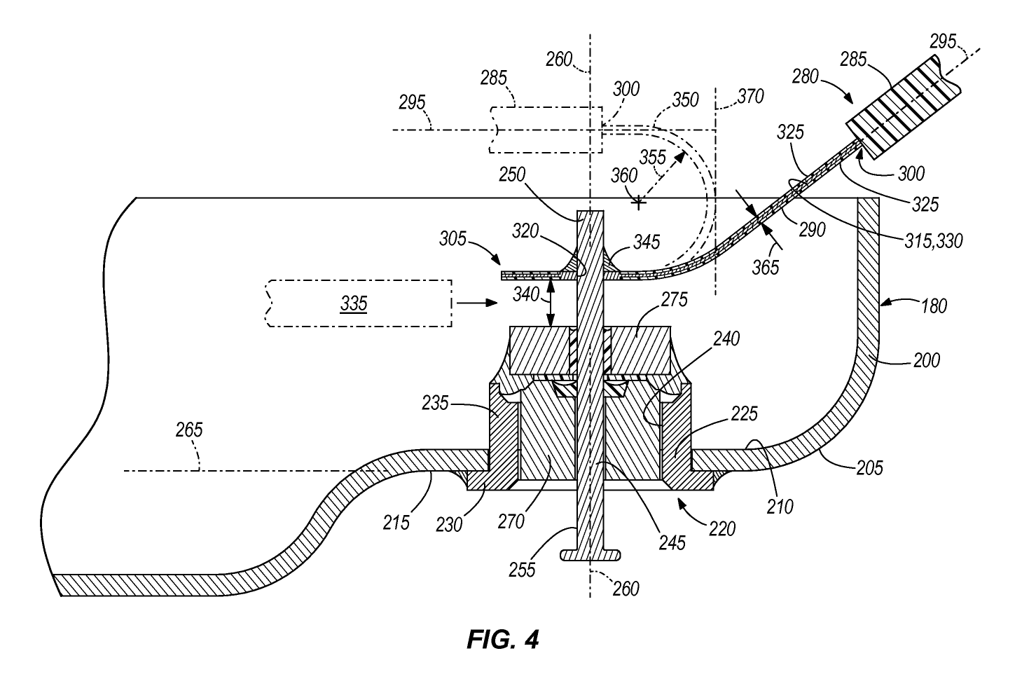

Referring to the cross-sectional view of FIG. 4, the first housing portion 180 includes a unitary wall 200 having an exterior surface 205 and an interior surface 210. The wall 200 defines a shelf 215. Referring back to FIG. 2, a plurality of feedthrough assemblies (“FTs”) 220 are coupled to exterior surface 205 on the shelf 215.

Referring again to FIG. 4, each FT 220 includes an FT body 225 that passes through the first housing portion 180, such that an FT body exterior portion 230 and a FT body interior portion 235 are defined. The FT body exterior portion 230 is welded to the exterior surface 205 of the first housing portion 180, such as by laser welding or by gold brazing. In other embodiments, the FT body 225 may be adhesively coupled to the first housing portion 180.

Each FT body 225 defines a plurality of apertures 240 extending through the exterior portion 230 and interior portion 235. An FT pin 245 extends through each aperture 240. In the illustrated embodiment, two of the FTs 220 are 8-pin FTs, while a third FT 220 is a 10-pin unit.

Referring to FIG. 4, each FT pin 245 defines an interior pin portion 250, extending from the FT housing interior portion 235, and an exterior pin portion 255, extending from the FT housing exterior portion 230. Each pin 245 is disposed along a pin axis 260 that is substantially perpendicular to a plane 265 of the first housing portion shelf 215.

An insulating layer 270 is disposed within the each aperture 240, between the pin 245 and the FT body 225. A capacitive filter 275 is disposed annularly about the interior pin portion 250. The capacitive filter 275 substantially reduces electrical and RF interference from the exterior of the IPG 115 to the interior of the IPG 115.

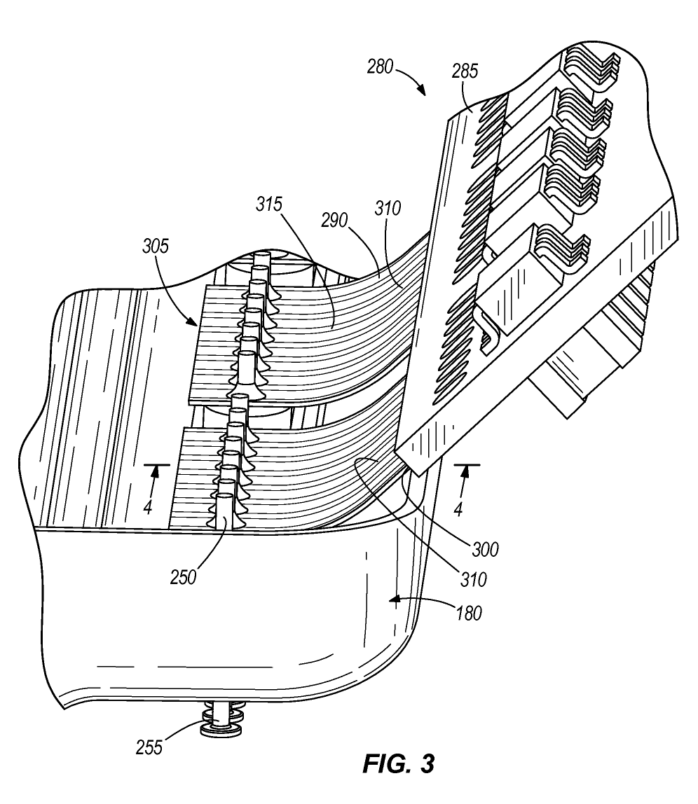

Referring now to FIG. 3, the IPG 115 includes a printed circuit board (PCB) 280 that contains control and pulse-generating circuitry. The printed circuit board 280 includes a substantially rigid portion 285, and a substantially flexible portion 290. Referring to FIG. 3, the substantially rigid portion 285 defines a plane 295. The substantially flexible portion 290 includes a proximal end 300, coupled to the substantially rigid portion 285, and a distal end 305. The proximal end 300 is substantially aligned with the plane 295 of the rigid portion 285.

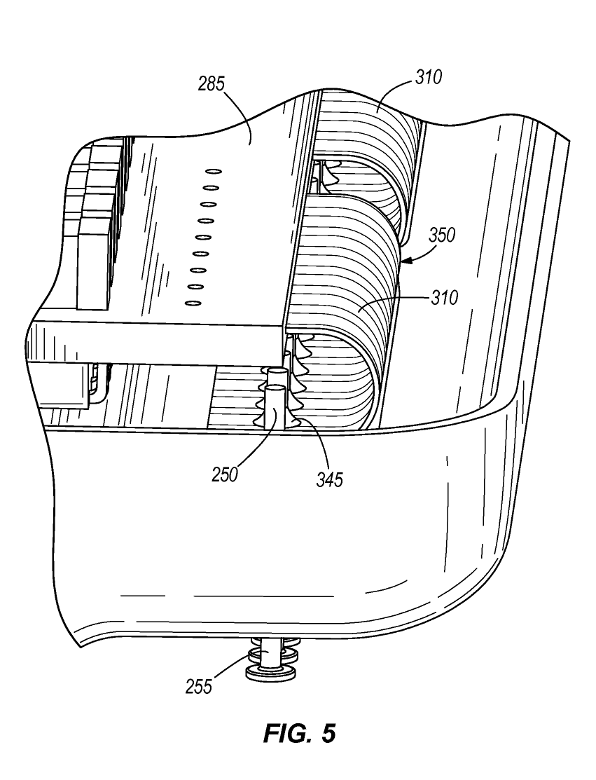

Referring to FIGS. 3 and 5, the flexible portion 290 is divided into sub-sections 310 which separately connect to the three FTs 220 within the IPG. The three sub-sections 310 accommodate variations in the position of the FTs 220. Referring to FIG. 3, the flexible portion 290 includes a plurality of conductive elements 315 extending from the proximal end 300 to the distal end 305. Referring to FIG. 4, each conductive element 315 defines an aperture 320 that is disposed adjacent the distal end 305. The apertures 320 are provided for hole soldering the conductive elements 315 to their corresponding FT pins 245.

The position and number of conductive elements 315 within each sub-section 310 generally corresponds to the position and number of the FT pins 245 of each FT 220. In the illustrated construction, two of the sub-sections 310 have eight conductive elements 315 for connection to the two 8-pin FTs 220. One of the sub-sections 310 has eleven conductive elements 315 for connection to the 10-pin FT. The eleventh conductive element 315 connects to the body 225 of the FT 220 in order to ground the IPG 115. The conductive elements 315 of the flexible portion 290 are an extension of conductors present in the rigid portion 285 of the PCB 280, so there is no intermediate joint. In other constructions, the flexible portion may comprise a plurality of ribbon-like sections, with one conductive element per ribbon-like section.

As best illustrated in FIG. 4, the conductive elements 315 are sandwiched between polyimide layers 325, with the conductive elements 315 disposed substantially along a geometric center 330 of the flexible portion 290. Positioning the conductive elements 315 along the geometric center 330 of the flexible portion 290 also substantially disposes the conductive elements 315 along a neutral strain axis, thereby increasing the fatigue life and mechanical-shock resistance of the PCB 280.

As shown in FIGS. 3 and 4, the PCB flexible portion 290 is initially coupled to the FT pins 245 with the PCB rigid portion 285 outside of the first housing portion 180. Keeping the rigid PCB portion 285 outside of the first housing portion 180 allows for better access to the FT pins 245, such as when hole-soldering the conductive elements 315 to the FT pins 245. As shown in FIG. 4, a soldering fixture 335 may be used to provide an offset 340 between solder joints 345 and the capacitive filter 275. The offset 340 substantially prevents thermal damage to the capacitive filter 275 during soldering.

Once the conductive elements 315 of the flexible portion 290 are soldered to the FT pins 245, a U-shaped bend 350 (FIG. 4) is formed in the flexible portion 290 by rotating the PCB rigid portion 285 into the first housing portion 180 (FIG. 5). As best illustrated in FIG. 4, the U-shaped bend 350 defines a radius 355 about an axis 360 that is substantially perpendicular to the pin axis 260. The radius 355 of the bend 345 is at least five times a thickness 365 of the flexible portion 290, in order to substantially maximize the reliability and fatigue life of the flexible portion 290. Once the U-shaped bend 350 is formed, the distal end 305 and the proximate end 300 of the flexible portion 290 are substantially parallel. At least one reference line 370 drawn tangent to the flexible portion 290 is substantially perpendicular to the plane 295 of the PCB rigid portion 280.

Thus, the invention provides, among other things, a useful implantable device and method of constructing the same. Various features and advantages of the invention are set forth in the following claims.

What is claimed is:

1. An implantable electronic device, comprising:
a housing having a wall including an interior surface and an exterior surface;
a feedthrough assembly including a body coupled to the housing and defining an aperture, and a pin at least partially disposed within the aperture and passing through the housing wall from the interior surface to the exterior surface such that the pin has an interior portion and an exterior portion;
a printed circuit board (PCB) having a substantially rigid portion defining a plane and a substantially flexible portion, the flexible portion having a distal end and a proximal end, the proximal end coupled to the substantially rigid portion, the flexible portion coupled to the pin interior portion adjacent the distal end, the flexible portion defining a bend between the proximal end and the distal end, with at least one line tangent to the flexible portion being substantially perpendicular to the plane.

2. The implantable electronic device of claim 1, wherein the flexible portion is coupled to the pin with a soldered joint.

3. The implantable electronic device of claim 1, wherein the soldered joint defines a standoff distance from the feedthrough body.

4. The implantable electronic device of claim 1, wherein the flexible portion comprises an electrically conductive element sandwiched between a first insulating layer and a second insulating layer.

5. The implantable electronic device of claim 4, wherein the conductive element is continuous with a conductor of the rigid portion.

6. The implantable electronic device of claim 4, wherein the conductive element defines an aperture for engaging the pin interior portion.

7. The implantable electronic device of claim 1, wherein the feedthrough body is welded to the housing.

8. The implantable electronic device of claim 1, wherein the feedthrough body is welded to the housing exterior surface.

9. The implantable electronic device of claim 1, wherein the feedthrough includes a capacitive filter disposed annularly about the feedthrough pin.

10. The implantable electronic device of claim 1, wherein the flexible portion defines a substantially U-shaped bend.

11. The implantable electronic device of claim 10, wherein the proximal end and the distal end are substantially parallel.

12. The implantable electronic device of claim 10, wherein the pin defines a pin axis that is substantially perpendicular to the PCB.

13. The implantable electronic device of claim 10, wherein the flexible portion includes a conductive layer disposed along a neutral strain axis of the bend.

14. The implantable electronic device of claim 13, wherein the conductive layer is sandwiched between a first insulating layer and a second insulating layer.

15. A method of assembling an implantable electronic device, the method comprising:
providing a housing having an interior surface and an exterior surface;
providing a feedthrough assembly including a body defining an aperture, and a pin at least partially disposed within the aperture and passing through body;
coupling the feedthrough assembly to the housing, the pin extending through the housing to define an interior portion and an exterior portion;
providing a printed circuit board (PCB) having a substantially rigid portion defining a plane and a substantially flexible portion, a proximal end of the flexible portion coupled to the rigid portion;
coupling a distal end of the flexible portion to the interior portion of the pin;
rotating the rigid portion about an axis substantially perpendicular to the pin; and
forming a bend between the proximal end and the distal end, with at least one line tangent to the flexible portion being substantially perpendicular to the plane.

16. The method of claim 15, wherein the act of coupling the feedthrough assembly to the housing includes welding the feedthrough body to an exterior surface of the housing.

17. The method of claim 15, wherein the act of forming a bend includes forming a U-shaped bend.

18. The method of claim 17, wherein the act of forming a bend results in the proximal end and the distal end being substantially parallel.

19. The method of claim 15, further comprising engaging an aperture of the flexible portion with the pin, such that the pin is received within the aperture.

20. The method of claim 19, further comprising soldering the flexible portion to the pin.
